(19) United States
(12) Patent Application Publication
(10) Pub. No.: US 2023/0329565 A1
(43) Pub. Date: Oct. 19, 2023

(54) DEVICE AND METHOD FOR DETECTING THE MEDICAL STATUS OF A PERSON

(71) Applicant: Franka Emika GmbH, München (DE)

(72) Inventors: Simon Haddadin, München (DE); Christoph Jähne, München (DE); Dmitrij Forssilow, München (DE); Saskia Golz, München (DE); Andreas Spenninger, Karlsfeld (DE); Simon Josef Gabl, München (DE); Carles Calafell Garcia, München (DE); Christian Mack, München (DE); Lukas Anton Hausperger, München (DE)

(21) Appl. No.: 17/798,452

(22) PCT Filed: Mar. 12, 2021
(86) PCT No.: PCT/EP2021/056288

§ 371 Date: Aug. 9, 2022

(30) Foreign Application Priority Data
Mar. 18, 2020 (DE) 10 2020 001 954.4

(51) Int. Cl. A61B 5/0205; A61B 5/00; A61B 5/15

(52) U.S. Cl. CPC A61B 5/02055; A61B 5/0077; A61B 5/150022; A61B 5/6835; A61B 5/0531

(57) ABSTRACT

A device to detect medical status of a person, including a robot manipulator to receive and handle effectors, each effector enabling a selected activity; a first unit to determine a current state Z_{RM}(t) of the robot manipulator and a current state of an effector; a second unit to determine a current position L_{KT,AKTk}(t) of a person's body part related to the selected activity in a working region of the robot manipulator; a third unit to determine a wrench KW(t) acting on the robot manipulator; a fourth unit to specify target data and permissible deviations for the selected activity; and a control unit to control the robot manipulator upon specification of the selected activity as a function of: Z_{RM}(t), L_{KT,AKTk}(t), KW(t), target data, and permissible deviations, such that when a permissible deviation is exceeded, the robot manipulator and the effector are placed into a safe state based on the selected activity.

### CROSS-REFERENCE TO RELATED APPLICATIONS

The present application is the U.S. National Phase of PCT/EP2021/056288, filed on 12 Mar. 2021, which claims priority to German Patent Application No. 10 2020 001 954.4, filed on 18 Mar. 2020, the entire contents of which are incorporated herein by reference.

### BACKGROUND

### Field

The invention relates to a device and a method for detecting the medical status of a person.

### Related Art

It is known that, in the case of persons who may have an infectious disease, in particular, a highly infectious disease, the medical personnel who are usually involved in determining the current medical status of such persons are sometimes exposed to a high risk of infection.

### SUMMARY

The object of the invention is to specify a device and a method for recording biosignals for detecting the medical status of a person which largely excludes potential risks of infection for medical personnel.

The invention results from the features of the independent claims. Advantageous refinements and embodiments are the subject matter of the dependent claims. Other features, applications, and advantages of the invention will be apparent from the following description and from the discussion of example embodiments of the invention, which are depicted in the figures.

A first aspect of the invention relates to a device for detecting the medical status of a person, wherein the device includes: at least a robot manipulator RM, which is designed and configured to exchangeably receive and handle a number N of different provided effectors EFFₙ at the distal end of the robot manipulator, where N≥1 and n=1, 2, . . . , N, wherein the effectors EFFₙ each enable an activity AKTₖ selected from the following list, where k=1, 2, . . . , K and K≥1:
sampling microbiological material from the person using provided swabs (AKT₁), applicator sticks (AKT₂), spatulas (AKT₃), biopsy needles (AKT₄);
sampling capillary blood from the person using provided capillaries (AKT₅);
recording medical parameters of the person: blood pressure (AKT₆), pulse (AKT₇), temperature (AKT₇), electrical skin resistance (AKT₈), electrical conductivity of the skin (AKT₉), local electrical potential measurement on the skin surface (AKT₁₀), oxygen saturation (AKT₁₁) by pulse oximetry, auscultation using an acoustic sensor (AKT₁₂);
capturing image data of the entire person (AKT₁₃) or of selected areas of the person, for example, the external auditory canal (AKT₁₄), the pharynx (AKT₁₅), specified skin areas (AKT₁₆), joints (AKT₁₇), eyes (AKT₁₈), feet (AKT₁₉), hands (AKT₁₇), head (AKT₁₈), neck (AKT₁₉), abdomen (AKT₂₀), and back (AKT₂₁).

The device further includes: a first unit for determining a current state Z_{RM} (t) of the robot manipulator RM and a current state Z_{EFFn}(t) of an effector EFFₙ currently arranged at the distal end thereof; a second unit for determining and/or mechanically specifying, in particular, in a customizable manner, the current position (position/orientation) L_{KT,AKTk}(t) of a person's body part KT associated with a selected activity AKTₖ in the working region of the robot manipulator; a third unit for determining a wrench KW(t) currently acting on the robot manipulator RM; a fourth unit which specifies, for each of the activities AKTₖ, a target position L_{KT,SOLL,AKTk} of the respectively assigned body part KT in the working region of the robot manipulator RM, a target movement of the robot manipulator RM BEWEG_{SOLL,RM,AKTk}, as well as target forces of interaction INT_{SOLL,AKTk} (BEWEG_{SOLL,RM,AKTk}) and target states of the effector Z_{EFFn,SOLL} (BEWEG_{SOLL,RM,AKTk}) between the robot manipulator RM (101) and the body part KT during execution of the target movement, as well as corresponding permissible deviations: ΔL_{KT,SOLL,AKTk,erlaubt}, ΔBEWEG_{SOLL,AKTk,erlaubt}, ΔINT_{SOLL,AKTk,erlaubt} (BEWEG_{SOLL,RM,AKTk}), and ΔZ_{EFFn,SOLL,erlaubt}(BEWEG_{SOLL,RM,AKTk}); and a control unit which is designed and configured to control the robot manipulator RM upon specification of an activity AKTₖ, as a function of: Z_{RM}(t), L_{KT,AKTk}(t), KW(t), L_{KT,SOLL,AKTk}(t), BEWEG_{SOLL,AKTk}, ΔL_{KT,SOLL,AKTk,erlaubt}, ΔBEWEG_{SOLL,AKTk,erlaubt}, ΔINT_{SOLL,AKTk,erlaubt}(BEWEG_{SOLL,AKTk}), and ΔZ_{EFFn,SOLL,erlaubt} (BEWEG_{SOLL,AKTn}), wherein, as soon as a current movement of the robot manipulator RM determined from the current states Z_{RM}(t), or a currently detected wrench KW(t), or a variable derived therefrom, or a current state Z_{EFF}(t) of the effector EFFₙ (102) currently arranged on the robot manipulator RM exceeds a correspondingly specified and/or learned permissible deviation, the robot manipulator RM and the effector EFFₙ currently arranged thereon are controllably placed into a safe state SZ_{RM} (AKTₙ), SZ_{EFFn} (AKTₙ) based on the currently selected activity AKTₙ, and furthermore to control the robot manipulator RM and the effector EFFₙ currently arranged thereon respectively such that images and/or medical parameters and/or samples ascertained while the activity AKTₖ is taking place are provided for further analysis at a respective interface.

In the present case, the term “medical status” is understood to mean at least the medical diagnoses resulting from the listed samples and recorded medical parameters and/or measurement data and/or image data.

In the present case, the robot manipulator RM is preferably an articulated manipulator with six or seven degrees of freedom. The robot manipulator RM can also be a continuum robot, a parallel robot, a linear robot, or any other movement apparatus. The robot manipulator RM is preferably force- and/or impedance- and/or admittance-controlled. It can also be current-controlled or flexibly controlled in some other way. It can also be position-controlled and have force feedback or a flexible end effector EFFₙ. The robot manipulator RM is preferably provided with an antibacterial, disinfecting coating.

The term activity “AKTₖ” is understood to mean at least those diagnostic activities that are designated as from AKT₁ to AKT₂₁. However, this list is not exhaustive, so that other diagnostic activities are also included in the concept of the invention.

The effectors EFFₙ for sampling microbiological material from the person are advantageously designed as grippers, so that the provided swabs, applicator sticks, spatulas, or biopsy needles can be advantageously gripped, handled according to the specified target movements, and placed in the provided lockable containers after the sampling has ended.

The effectors EFFₙ for sampling capillary blood from the person advantageously include, on the one hand, a device for puncturing the skin and a corresponding gripper with which the provided capillary tubes can be gripped, handled according to the specified target movement, and placed in the provided lockable containers after the sampling has ended.

The effectors EFFₙ for detecting medical parameters of the person advantageously include appropriate sensors for measuring blood pressure, heart rate, temperature, electrical skin resistance, electrical conductivity of the skin, local electrical potentials on the skin surface, oxygen saturation (in particular by pulse oximetry), and for listening to the lungs, the heart, etc. (auscultation). This list is not exhaustive. Thus, further activities AKTₖ such as the recording of medical parameters and data through the use of ultrasonic sensors, laser sensors, X-ray sensors, etc. are included in the concept of the invention.

The effectors EFFₙ for capturing image data advantageously include corresponding imaging sensors, such as camera sensors, infrared sensors, laser sensors, ultrasonic sensors, X-ray sensors, magnetic resonance sensors, etc.

The effectors EFFₙ are advantageously coupled manually or automatically to the distal end of the robot manipulator RM as a function of a selected activity AKTₖ. An effector EFFₙ can advantageously have several different sensors and/or have instruments for performing different activities AKTₖ. The robot manipulator RM and the effector EFF currently arranged on the robot manipulator RM are controlled by the control unit.

The term “state Z_{RM}(t) of the robot manipulator RM” is understood in a broad sense here. This includes, in particular, the dynamic and electrical state of the robot manipulator RM.

The term “state Z_{EFFn}(t) of the effector EFFₙ” is also understood broadly in the present case. This includes, in particular, the dynamic and electrical state of the effector EFFₙ.

In the present case, the state Z_{RM}(t) of the robot manipulator RM and the state Z_{EFFn}(t) of the effector EFFₙ are determined by the first unit. For this purpose, corresponding information and data from the robot manipulator RM and the respective effector EFFₙ are advantageously transmitted to the first unit. Alternatively or additionally, information and data from external sensors, observers, etc. can be used for this.

The second unit is used firstly to determine the current position (i.e., the position and orientation) L_{KT,AKTk}(t) of a person's body part KT associated with a selected activity AKTₖ in the working area of the robot manipulator RM and/or secondly to mechanically specify, in a customizable manner, a current position L_{KT,AKTk}(t) of a person's body part KT associated with a selected activity AKTₖ in the working area of the robot manipulator RM.

In the first case, the second unit includes sensors (in particular, imaging sensors: e.g., camera sensor, video sensor, infrared sensor, ultrasonic sensor, laser scanner, etc.) and/or distance-measuring sensors (in particular ultrasonic sensors, capacitive sensors, laser sensors, etc.) via which a 3D position and 3D orientation of the body part KT to be analyzed is preferably possible. These sensors thus actively detect the current position of the body part KT. In this case, the first unit thus includes a corresponding processor on which a corresponding evaluation program (app) runs.

In the second case, the unit preferably includes a mechanical contact or guide device with which the person can position a body part KT to be examined on the mechanical contact or guide device with a predetermined position and orientation. This contact or guide device is preferably made of a material that can be easily disinfected, or it is replaced manually or automatically as a consumable before each corresponding examination activity AKTₖ and is thus renewed.

The contact or guide device can be designed, for example, in such a way that the person places their nose or their mouth opening or one of their ear openings in the position and orientation predetermined by the design and positioning of the device. The position and orientation specified by the contact or guide device can preferably be individualized and can preferably be adjusted manually by the person or, in the case of interaction between the first and second case, it can be automatically individualized (positioned) based on the optical conditions of the person recorded by sensors.

The third unit determines wrenches KW(t), i.e., forces and/or torques, currently acting on the robot manipulator RM. The third unit is advantageously connected to at least one force sensor or a contact detection device on the robot manipulator or on the effector EFFₙ. The third unit thus determines the forces of interaction and interaction torques during an interaction between the robot manipulator RM or the effector EFFₙ currently arranged distally thereon and the person when performing an activity AKTₖ. In order to rule out injuries or unnecessary pain to the person when performing an activity AKTₖ, wrenches that occur must be determined with high sensitivity and reliability (robustness). For this purpose, the third unit includes a corresponding processor on which a corresponding external program (app) runs.

For each of the activities AKTₖ, the fourth unit provides information and data on the target position L_{KT,SOLL,AKTk} of the associated body part KT in the working area of the robot manipulator RM, on the target movement of the robot manipulator RM BEWEG_{SOLL,RM,AKTk}, and on the target forces of interaction INT_{SOLL,AKTk}(BEWEG_{SOLL,RM,AKTk}) and target states of the effector Z_{EFFn,SOLL}(BEWEG_{SOLL,RM,AKTk}) between the robot manipulator RM (101) and the body part KT during the execution of the target movement, as well as associated permissible deviations: ΔL_{KT,SOLL,AKTk,erlaubt}, ΔBEWEG_{SOLL,AKTk,erlaubt}, ΔINT_{SOLL,AKTk,erlaubt}(BEWEG_{SOLL,RM,AKTk}), and ΔZ_{EFFn,SOLL,erlaubt}(BEWEG_{SOLL,RM,AKTk}). The fourth unit advantageously includes a corresponding data memory and a corresponding processor on which corresponding software (app) runs.

The proposed device enables the medical status of persons to be detected without the need for medical personnel. The proposed device can be operated fully autonomously, partially autonomously, or remotely. The proposed device is preferably mobile and can therefore be easily brought to medical crisis zones, in particular.

One advantageous refinement of the proposed device is characterized in that the device has a first data interface, via which individual medical information INFO_{PERS} about the person to be examined includes one or more items of information from the following (non-exhaustive) list:
pre-existing illnesses;
allergies;
familial illnesses;
current medications;
medical risk factors;
previous diagnoses; and
current diagnostic query.

In this refinement, the device also includes an analysis unit, which is designed and configured to specify one or more current activities AKTₖ to be carried out in succession, depending on the individual medical information INFO_{PERS} provided: AKTₖ=AKTₖ(INFO_{PERS}). For this purpose, the first data interface advantageously includes a card reader, wherein the individual medical information INFO_{PERS} is stored on a chip card (e.g., health insurance card G2) and the card reader is designed and configured to read the chip card. The analysis unit also advantageously uses a neural network and a self-learning algorithm to determine AKTₖ(INFO_{PERS}). In particular, this enables a more targeted diagnosis planning based on available medical history information and thus a corresponding selection of activities AKTₖ to be performed.

One advantageous refinement of the proposed device is characterized in that a second data interface is present via which the recorded medical parameters and/or image data can be transmitted to a computer in a data network. Advantageously, the second data interface is also designed to receive data or instructions from the computer or from a control unit, which data or instructions are sent to an optical and/or acoustic output unit of the device (and relate, for example, to instructions to the person) and/or which enable the robot manipulator RM to be controlled from the computer or the control unit.

One advantageous refinement of the proposed device is characterized in that the robot manipulator RM is integrated into a sealable, advantageously climate-controlled, room which has a mechanism for killing microbiological germs in the room and on the objects located therein, wherein this mechanism is automatically activated after the detection of the medical status of the person has ended, once the person has left the room, no other person is in the room, and the room is closed up. The room is advantageously sealed in such a way that microbiological germs cannot penetrate the room from the outside if the room is closed up. The mechanism advantageously includes a UV light source and/or chemicals that can be spread into the room and/or an ultrasound source. When the mechanism is activated, the amount of the application of chemicals or the intensity of the UV light source or the ultrasound source is set in such a way that the room is sufficiently sterile before the next person is examined.

One advantageous refinement of the proposed device is characterized in that the device has an output unit for outputting optical and/or acoustic instructions to the person, wherein the instructions are determined and output as a function of the determined current position (position/orientation) L_{KT,AKTk}(t) of the person's body part KT associated with the selected activity AKTₖ. The output unit thus serves, in particular, to guide and inform the person while their medical status is being determined.

One advantageous refinement of the proposed device is characterized in that the robot manipulator RM is arranged in a room separated from the person by a partition, wherein the partition has an opening, and wherein the control unit is designed and configured in such a way that the robot manipulator RM only guides a distal end of a respective swab or a respective applicator stick or a respective swab spatula or a respective biopsy needle through the opening into the person's body part KT to be examined, in order to get a sample of the microbiological material of the person.

This room is also advantageously sealed in such a way that microbiological germs cannot penetrate the room from the outside if the room is closed up. The room advantageously includes a mechanism with a UV light source and/or chemicals that can be spread into the room and/or an ultrasound source. When the mechanism is activated, the amount of the application of chemicals or the intensity of the UV light source or the ultrasound source is set in such a way that the room remains sufficiently sterile.

Advantageously, the opening on the side of the partition facing away from the room has a contact device for the supported and/or guided contact of the body part to be examined. Advantageously, the opening is designed in such a way that predefined movement restrictions of a guided-through distal end of a respective swab or a respective applicator stick or a respective swab spatula or a respective biopsy needle are predefined. This creates an additional safety framework, which, in particular, mechanically restricts the range of movement of a distal end guided through the opening sufficiently well enough such that injuries to the body part being examined can be excluded. The partition advantageously has a mechanism for disinfecting the contact device.

A further aspect of the invention relates to a method of detecting the medical status of a person, using a device including at least one robot manipulator RM, which is designed and configured at the distal end thereof for interchangeably receiving and handling a number N of different provided effectors EFFₙ, where N≥1 and n=1, 2, . . . , N, wherein the effectors EFFₙ each enable an activity AKTₖ selected from the following list, where k=1, 2, . . . , K and K≥1:
sampling microbiological material from the person using provided swabs (AKT₁), applicator sticks (AKT₂), spatulas (AKT₃), biopsy needles (AKT₄);
sampling capillary blood from the person using provided capillaries (AKT₅);
recording medical parameters of the person: blood pressure (AKT₆), pulse (AKT₇), temperature (AKT₇), electrical skin resistance (AKT₈), electrical conductivity of the skin (AKT₉), local electrical potential measurement on the skin surface (AKT₁₀), oxygen saturation (AKT₁₁) by pulse oximetry, auscultation using an acoustic sensor (AKT₁₂); and
capturing image data of the entire person (AKT₁₃) or of selected areas of the person, for example, the external auditory canal (AKT₁₄), the pharynx (AKT₁₅), specified skin areas (AKT₁₆), joints (AKT₁₇), eyes (AKT₁₈), feet (AKT₁₉), hands (AKT₁₇), head (AKT₁₈), neck (AKT₁₉), abdomen (AKT₂₀), and back (AKT₂₁).

The proposed method includes: In one step, a determination of a current state Z_{RM}(t) of the robot manipulator RM and a current state Z_{EFFn}(t) of an effector EFFₙ currently arranged at the distal end thereof. In a further step, there is a determination and/or in particular customizable, mechanical specification of a current position (position/orientation) L_{KT,AKTk}(t) of a person's body part KT associated with a selected activity AKTₖ in the working area of the robot manipulator RM. In a further step, there is a determination of a wrench KW(t) currently acting on the robot manipulator RM. In a further step, the following is specified for each of the activities AKTₖ: a target position L_{KT,SOLL,AKTk} of the respectively associated body part KT in the working region of the robot manipulator RM, a target movement of the robot manipulator RM BEWEG_{SOLL,RM,AKTk}, as well as the target forces of interaction INT_{SOLL,AKTk}(BEWEG_{SOLL,RM,AKTk}) and target states of the effector Z_{EFFn,SOLL}(BEWEG_{SOLL,RM,AKTk}) between the robot manipulator RM (101) and the body part KT during the execution of the target movement, as well as associated permissible deviations: ΔL_{KT,SOLL,AKTk,erlaubt}, ΔBEWEG_{SOLL,AKTk,erlaubt}, ΔINT_{SOLL,AKTk,erlaubt}(BEWEG_{SOLL,RM,AKTn}), and ΔZ_{EFFFn,SOLL,erlaubt}(BEWEG_{SOLL,RM,AKTk}). In a further step, upon specification of an activity AKTₖ, the robot manipulator RM is controlled as a function of: Z_{RM}(t), L_{KT,AKTk}(t), KW(t), L_{KT,SOLL,AKTk}(t), BEWEG_{SOLL,AKTk}, ΔL_{KT,SOLL,AKTk,erlaubt}, ΔBEWEG_{SOLL,AKTk,erlaubt}, ΔINT_{SOLL,AKTk,erlaubt}(BEWEG_{SOLL,AKTk}), and ΔZ_{EFFn,SOLL,erlaubt}(BEWEG_{SOLL,AKTn}), wherein, as soon as a current movement of the robot manipulator RM determined from the current state Z_{RM}(t) or a currently detected wrench KW(t) or a variable derived therefrom or a current state Z_{EFF}(t) of the effector EFFₙ currently arranged on the robot manipulator RM exceeds a correspondingly predetermined and/or learned permissible deviation, the robot manipulator RM and the effector EFFₙ currently arranged thereon are controllably placed in a safe state SZ_{RM}(AKTₙ), SZ_{EFFn}(AKTₙ), as a function of the currently selected activity AKTₙ. Furthermore, the robot manipulator RM (101) and the effector EFFₙ (102) currently arranged thereon are controlled in such a way that images and/or medical parameters and/or samples ascertained while the activity AKTₖ is taking place are provided for further evaluation at a respective interface.

One advantageous refinement of the proposed method is characterized in that one or more items of individual medical information INFO_{PERS} about the person to be examined are provided from the following list via a first data interface:
pre-existing illnesses;
allergies;
familial illnesses;
current medications;
medical risk factors;
previous diagnoses;
current diagnostic query; and
one or more activities AKTₖ to be carried out one after the other are specified depending on the individual medical information INFO_{PERS} provided: AKTₖ=AKTₖ(INFO_{PERS}).

One advantageous refinement of the proposed method is characterized in that the first data interface comprises a card reader, wherein the individual medical information INFO_{PERS} is stored on a chip card (e.g., health insurance card G2) and in that the information INFO_{PERS} is read and made available by the card reader.

One advantageous refinement of the proposed method is characterized in that a neural network and a self-learning algorithm are used to determine AKTₖ(INFO_{PERS}).

One advantageous refinement of the proposed method is characterized in that the recorded medical parameters and/or image data are transmitted to a computer in a data network via a second data interface.

One advantageous refinement of the proposed method is characterized in that the second data interface receives data from the computer, which data are output to an optical and/or acoustic output unit of the device and/or which enable the robot manipulator RM to be controlled from the computer.

One advantageous refinement of the proposed method is characterized in that the robot manipulator RM is integrated into a sealable, climate-controlled room which has a mechanism for killing microbiological germs in the room and on the objects located therein, wherein this mechanism is automatically activated after the detection of the medical status of the person has ended, once the person has left the room, no other person is in the room, and the room is closed up.

One advantageous refinement of the proposed method is characterized in that the mechanism comprises a UV light source and/or chemicals that can be spread into the room and/or an ultrasound source.

One advantageous refinement of the proposed method is characterized in that an output unit is present for outputting optical and/or acoustic instructions to the person, wherein the instructions are determined and output as a function of the determined current position (position/orientation) L_{KT,AKTk}(t) of the person's body part KT associated with the selected activity AKTₖ.

One advantageous refinement of the proposed method is characterized in that the robot manipulator RM is arranged in a room separated from the person by a partition, wherein the partition has an opening, and wherein the control unit controls the robot manipulator RM in such a way that only a distal end of a respective swab or a respective applicator stick or a respective swab spatula or a respective biopsy needle is guided through the opening into the person's body part KT to be examined, in order to get a sample of the microbiological material of the person.

One advantageous refinement of the proposed method is characterized in that the opening on the side of the partition facing away from the room has a contact device for the supported and/or guided contact of the body part to be examined.

One advantageous refinement of the proposed method is characterized in that the contact device is automatically disinfected by a disinfection mechanism after each use.

With regard to advantages and advantageous refinements of the proposed method, reference is made to the statements on the proposed device, which are to be transferred correspondingly and analogously.

Further advantages, features, and details will be apparent from the following description, in which—possibly with reference to the drawings—at least one example embodiment is described in detail. The same, similar, and/or functionally identical parts are provided with the same reference numerals.

### BRIEF DESCRIPTION OF THE DRAWINGS

In the drawings:

FIG. 1 shows a highly schematized structure of a proposed device; and

FIG. 2 shows a highly schematized flowchart of a proposed method.

### DETAILED DESCRIPTION

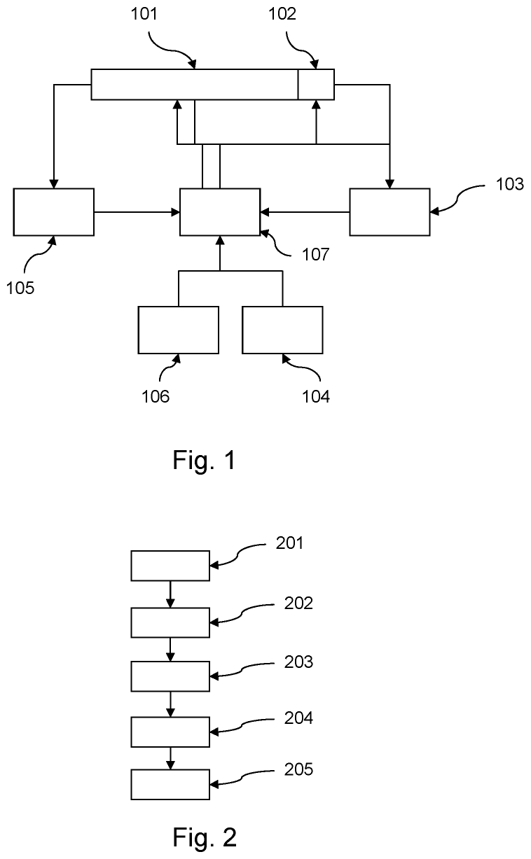

FIG. 1 shows a highly schematized structure of a proposed device for detecting the medical status of a person, wherein the device includes: at least one force-controlled and/or impedance-controlled multi-axis robot manipulator RM 101, which is designed and configured at the distal end thereof for interchangeably receiving and handling a number N of different provided effectors EFFₙ 102, where N≥1 and n=1, 2, . . . , N, wherein the effectors EFFₙ 102 each enable an activity AKTₖ selected from the following list, where k=1, 2, . . . , K and K≥1:
sampling microbiological material from the person using provided swabs (AKT₁), applicator sticks (AKT₂), swab spatulas (AKT₃), biopsy needles (AKT₄);
sampling capillary blood from the person using provided capillaries (AKT₅);
recording medical parameters of the person: blood pressure (AKT₆), pulse (AKT₇), temperature (AKT₇), electrical skin resistance (AKT₈), electrical conductivity of the skin (AKT₉), local electrical potential measurement on the skin surface (AKT₁₀), oxygen saturation (AKT₁₁) by pulse oximetry, auscultation using an acoustic sensor (AKT₁₂); and
capturing image data of the entire person (AKT₁₃) or of selected areas of the person, for example the external auditory canal (AKT₁₄), the pharynx (AKT₁₅), specified skin areas (AKT₁₆), joints (AKT₁₇), eyes (AKT₁₈), feet (AKT₁₉), hands (AKT₁₇), head (AKT₁₈), neck (AKT₁₉), abdomen (AKT₂₀), and back (AKT₂₁);

a first unit 103 for determining a current state Z_{RM}(t) of the robot manipulator RM 101 and a current state Z_{EFFn}(t) of an effector EFFₙ 102 currently arranged at the distal end thereof; a second unit 104 for determining and/or mechanically specifying, in particular, in a customizable manner, the current position L_{KT,AKTk}(t) of a person's body part KT associated with a selected activity AKTₖ in the working region of the robot manipulator RM 101; a third unit 105 for determining a wrench KW(t) currently acting on the robot manipulator RM 101; a fourth unit 106 which specifies, for each of the activities AKTₖ, a target position L_{KT,SOLL,AKTk} of the respectively assigned body part KT in the working region of the robot manipulator RM, a target movement of the robot manipulator RM BEWEG_{SOLL,RM,AKTk}, as well as target forces of interaction INT_{SOLL,AKTn}(BEWEG_{SOLL,RM,AKTk}) and target states of the effector Z_{EFFn,SOLL}(BEWEG_{SOLL,RM,AKTk}) between the robot manipulator RM 101 and the body part KT during execution of the target movement, as well as corresponding permissible deviations: ΔL_{KT,SOLL,AKTk,erlaubt}, ΔBEWEG_{SOLL,AKTk,erlaubt}, ΔINT_{SOLL,AKTk,erlaubt}(BEWEG_{SOLL,RM,AKTk}), and ΔZ_{EFFn,SOLL,erlaubt}(BEWEG_{SOLL,RM,AKTk}); and a control unit 107 which is designed and configured to control the robot manipulator RM 101 upon specification of an activity AKTₖ, as a function of: Z_{RM}(t), L_{KT,AKTk}(t), KW(t), L_{KT,SOLL,AKT}(t), BEWEG_{SOLL,AKTk}, ΔL_{KT,SOLL,AKTk,erlaubt}, ΔBEWEG_{SOLL,AKTk,erlaubt}, ΔINT_{SOLL,AKTk,erlaubt}(BEWEG_{SOLL,AKTk}), and ΔZ_{EFFn,SOLL,erlaubt}(BEWEG_{SOLL,AKTn}), wherein, as soon as a current movement of the robot manipulator RM 101 determined from the current state Z_{RM}(t), or a currently detected wrench KW(t), or a variable derived therefrom, or a current state Z_{EFF}(t) of the effector EFFₙ 102 currently arranged on the robot manipulator RM 101 exceeds a correspondingly specified and/or learned permissible deviation, the robot manipulator RM 101 and the effector EFFₙ 102 currently arranged thereon are controllably placed into a safe state SZ_{RM}(AKTₙ), SZ_{EFFn}(AKTₙ) based on the currently selected activity AKTₙ, to control the robot manipulator RM 101 and the effector EFFₙ 102 currently arranged thereon respectively such that images and/or medical parameters and/or samples ascertained while the activity AKTₖ is taking place are provided for further analysis at a respective interface.

FIG. 2 shows a highly schematized flowchart of a proposed method for detecting the medical status of a person, using a device including at least one force-controlled and/or impedance-controlled multi-axis robot manipulator RM 101, which is designed and configured at the distal end thereof for interchangeably receiving and handling a number N of different provided effectors EFFₙ 102, where N≥1 and n=1, 2, . . . , N, wherein the effectors EFFₙ 102 each enable an activity AKTₖ selected from the following list, where k=1, 2, . . . , K and K≥1:
sampling microbiological material from the person using provided swabs (AKT₁), applicator sticks (AKT₂), swab spatulas (AKT₃), biopsy needles (AKT₄);
sampling capillary blood from the person using provided capillaries (AKT₅);
recording medical parameters of the person: blood pressure (AKT₆), pulse (AKT₇), temperature (AKT₇), electrical skin resistance (AKT₈), electrical conductivity of the skin (AKT₉), local electrical potential measurement on the skin surface (AKT₁₀), oxygen saturation (AKT₁₁) by pulse oximetry, auscultation using an acoustic sensor (AKT₁₂);
capturing image data of the entire person (AKT₁₃) or of selected areas of the person, for example the external auditory canal (AKT₁₄), the pharynx (AKT₁₅), specified skin areas (AKT₁₆), joints (AKT₁₇), eyes (AKT₁₈), feet (AKT₁₉), hands (AKT₁₇), head (AKT₁₈), neck (AKT₁₉), abdomen (AKT₂₀), and back (AKT₂₁), wherein the method includes:

determining 201 a current state Z_{RM}(t) of the robot manipulator RM 101 and a current state Z_{EFFn}(t) of an effector EFFₙ 102 currently arranged at the distal end thereof; determining 202 and/or mechanically specifying, in particular, in a customizable manner, the current position L_{KT,AKTk}(t) of a person's body part KT associated with a selected activity AKTₖ in the working region of the robot manipulator RM 101; determining 203 a wrench KW(t) currently acting on the robot manipulator RM 101; specifying 204, for each of the activities AKTₖ, a target position L_{KT,SOLL,AKTk} of the respectively assigned body part KT in the working region of the robot manipulator RM, a target movement of the robot manipulator RM BEWEG_{SOLL,RM,AKTk}, as well as target forces of interaction INT_{SOLL,AKTk}(BEWEG_{SOLL,RM,AKTk}) and target states of the effector Z_{EFFn,SOLL}(BEWEG_{SOLL,RM,AKTk}) between the robot manipulator RM 101 and the body part KT during execution of the target movement, as well as corresponding permissible deviations: ΔL_{KT,SOLL,AKTk,erlaubt}, ΔBEWEG_{SOLL,AKTk,erlaubt}, ΔINT_{SOLL,AKTk,erlaubt}(BEWEG_{SOLL,RM,AKTk}), and ΔZ_{EFFn,SOLL,erlaubt}(BEWEG_{SOLL,RM,AKTk}); and controlling 205 the robot manipulator RM 101 upon specification of an activity AKTₖ, as a function of: Z_{RM}(t), L_{KT,AKTk}(t), KW(t), L_{KT,SOLL,AKTk}(t), BEWEG_{SOLL,AKTk}, ΔL_{KT,SOLL,AKTk,erlaubt}, ΔBEWEG_{SOLL,AKTk,erlaubt}, ΔINT_{SOLL,AKTk,erlaubt}(BEWEG_{SOLL,AKTk}), and ΔZ_{EFFn,SOLL,erlaubt}(BEWEG_{SOLL,AKTn}), wherein, as soon as a current movement of the robot manipulator RM 101 determined from the current state Z_{RM}(t), or a currently detected wrench KW(t), or a variable derived therefrom, or a current state Z_{EFF}(t) of the effector EFFₙ 102 currently arranged on the robot manipulator RM 101 exceeds a correspondingly specified and/or learned permissible deviation, the robot manipulator RM 101 and the effector EFFₙ 102 currently arranged thereon are controllably placed into a safe state SZ_{RM}(AKTₙ), SZ_{EFFn}(AKTₙ) based on the currently selected activity AKTₙ, and furthermore, the robot manipulator RM 101 and the effector EFFₙ 102 currently arranged thereon respectively are controlled such that images and/or medical parameters and/or samples ascertained while the activity AKTₖ is taking place are provided for further analysis at a respective interface.

Although the invention has been further illustrated and described in detail by way of preferred example embodiments, the invention is not limited by the disclosed examples, and other variations can be derived therefrom by a person skilled in the art without departing from the scope of protection of the invention. It is therefore clear that a multitude of possible variations exists. It is also clear that example embodiments are really only examples, which are not to be construed in any way as limiting the scope, applicability, or configuration of the invention. Rather, the foregoing description and description of the figures enable a person skilled in the art to implement the example embodiments, and such person may make various changes knowing the disclosed inventive concept, for example with respect to the function or arrangement of individual elements cited in an example embodiment, without departing from the scope as defined by the claims and their legal equivalents, such as a more extensive explanation in the description.

### LIST OF REFERENCE NUMERALS

101 Robot manipulator RM
102 Effectors EFFₙ
103 First unit
104 Second unit
105 Third unit
106 Fourth unit
107 Control unit
108 First data interface
109 Analysis unit
110 Second data interface
201-204 Method steps

1. A device to detect a medical status of a person, wherein the device comprises:
at least one robot manipulator RM configured at a distal end thereof to interchangeably receive and handle a number N of different provided effectors EFFₙ, where N≥1 and n=1, 2, . . . , N, wherein the effectors EFFₙ each enable an activity AKTₖ selected from a following list, where k=1, 2, . . . , K and K≥1:
i. sampling microbiological material from the person using provided swabs, applicator sticks, swab spatulas, biopsy needles;
ii. sampling capillary blood from the person using provided capillaries;
iii. recording medical parameters of the person: blood pressure, pulse, temperature, electrical skin resistance, electrical conductivity of the skin, local electrical potential measurement on the skin surface, oxygen saturation, auscultation using an acoustic sensor;
iv. capturing image data of the person in person's entirety or of selected areas of the person;
a first unit configured to determine a current state Z_{RM}(t) of the robot manipulator RM and a current state Z_{EFFn}(t) of an effector EFFₙ currently arranged at the distal end thereof;
a second unit configured to determine and/or mechanically specify, in a particularly customizable manner, a current position L_{KT,AKTk}(t) of a person's body part KT associated with a selected activity AKTₖ in a working region of the robot manipulator RM;
a third unit configured to determine a wrench KW(t) currently acting on the robot manipulator RM;
a fourth unit configured to specify for each of the activities AKTₖ, information and data on a target position L_{KT,SOLL,AKTk} of a respectively associated body part KT in the working region of the robot manipulator RM, on a target movement of the robot manipulator RM BEWEG_{SOLL,RM,AKTk}, and on target forces of interaction INT_{SOLL,AKTk}(BEWEG_{SOLL,RM,AKTk}) and target states of the effector Z_{EFFn,SOLL}(BEWEG_{SOLL,RM,AKTk}) between the robot manipulator RM and the body part KT during execution of the target movement, as well as associated permissible deviations: ΔL_{KT,SOLL,AKTk,erlaubt}, ΔBEWEG_{SOLL,AKTk,erlaubt}, ΔINT_{SOLL,AKTk,erlaubt}(BEWEG_{SOLL,RM,AKTk}), and ΔZ_{EFFn,SOLL,erlaubt}(BEWEG_{SOLL,RM,AKTk}); and
a control unit configured to control the robot manipulator RM upon specification of an activity AKTₖ, as a function of: Z_{RM}(t), L_{KT,AKTk}(t), KW(t), L_{KT,SOLL,AKTk}(t), BEWEG_{SOLL,AKTk}, ΔL_{KT,SOLL,AKTk,erlaubt}, ΔBEWEG_{SOLL,AKTk,erlaubt}, ΔINT_{SOLL,AKTk,erlaubt}(BEWEG_{SOLL,AKTk}), and ΔZ_{EFFn,SOLL,erlaubt}(BEWEG_{SOLL, AKTn}), wherein, as soon as a current movement of the robot manipulator RM determined from the current state Z_{RM}(t) or a currently detected wrench KW(t) or a variable derived therefrom or a current state Z_{EFF}(t) of the effector EFFₙ currently arranged on the robot manipulator RM exceeds a correspondingly predetermined and/or learned permissible deviation, the robot manipulator RM and the effector EFFₙ currently arranged thereon are controllably placed in a safe state SZ_{RM}(AKTₙ), SZ_{EFFn}(AKTₙ) as a function of the currently selected activity AKTₙ, and furthermore to control the robot manipulator RM and the effector EFF currently arranged thereon in such a way that images and/or medical parameters and/or samples ascertained while the activity AKTₖ is taking place are provided for further evaluation at a respective interface.

2. The device according to claim 1, wherein the device comprises:
a first data interface, via which individual medical information INFO_{PERS} about the person to be examined includes one or more items from a following list:
pre-existing illnesses;
allergies;
familial illnesses;
current medications;
medical risk factors;
previous diagnoses;
current diagnostic query; and
an analysis unit configured to specify one or more current activities AKTₖ to be carried out in succession, depending on the individual medical information INFO_{PERS} provided: AKTₖ=AKTₖ(INFO_{PERS}).

3. The device according to claim 2, wherein the first data interface comprises a card reader, wherein the individual medical information INFO_{PERS} is stored on a chip card and the card reader is configured to read the chip card.

4. The device according to claim 1, wherein the device comprises a second data interface configured to transmit recorded medical parameters and/or image data to a computer in a data network.

5. The device according to claim 4, wherein the second data interface is configured to receive data from the computer or the control unit, wherein the data are output at an optical output unit and/or acoustic output unit of the device and/or which enable the robot manipulator RM to be controlled from the computer.

6. The device according to claim 1, wherein the robot manipulator RM is integrated into a sealable, climate-controlled room which includes a mechanism to kill microbiological germs in the room and on objects located therein, wherein the mechanism is automatically activated after detection of the medical status of the person has ended, once the person has left the room, no other person is in the room, and the room is closed.

7. The device according to claim 6, wherein the mechanism comprises a UV light source and/or chemicals capable of being spread into the room and/or an ultrasound source.

8. The device according to claim 1, wherein the robot manipulator RM is arranged in a room separated from the person by a partition, wherein the partition includes an opening, and wherein the control unit is configured such that the robot manipulator RM only guides a distal end of a respective swab or a respective applicator stick or a respective swab spatula or a respective biopsy needle through the opening into the person's body part KT to be examined, in order to get a sample of the microbiological material of the person.

9. The device according to claim 8, wherein the opening on a side of the partition facing away from the room includes a contact device for supported and/or guided contact of the body part to be examined.

10. (canceled)

11. The device according to claim 1, wherein the selected areas of the person include at least one of external auditory canal, pharynx, specified skin areas, joints, eyes, feet, hands, head, neck, abdomen, and back.

12. A method of detecting the medical status of a person, wherein the method comprises:
using at least one robot manipulator RM configured at a distal end thereof to interchangeably receive and handle a number N of different provided effectors EFFₙ, where N≥1 and n=1, 2, . . . , N, wherein the effectors EFFₙ each enable an activity AKTₖ selected from a following list, where k=1, 2, . . . , K and K≥1:
i. sampling microbiological material from the person using provided swabs, applicator sticks, swab spatulas, biopsy needles;
ii. sampling capillary blood from the person using provided capillaries;
iii. recording medical parameters of the person: blood pressure, pulse, temperature, electrical skin resistance, electrical conductivity of the skin, local electrical potential measurement on the skin surface, oxygen saturation by pulse oximetry, auscultation using an acoustic sensor;
iv. capturing image data of the person in person's entirety or of selected areas of the person;
determining a current state Z_{RM}(t) of the robot manipulator RM and a current state Z_{EFFn}(t) of an effector EFFₙ currently arranged at the distal end thereof;
determining and/or mechanically specifying, in a particularly customizable manner, a current position L_{KT,AKTk}(t) of a person's body part KT associated with a selected activity AKTₖ in a working region of the robot manipulator RM;
determining a wrench KW(t) currently acting on the robot manipulator RM;
specifying for each of the activities AKTₖ, a target position L_{KT,SOLL,AKTk} of a respectively associated body part KT in the working region of the robot manipulator RM, on a target movement of the robot manipulator RM BEWEG_{SOLL,RM,AKTk}, and target forces of interaction INT_{SOLL,AKTk}(BEWEG_{SOLL,RM,AKTk}) and target states of the effector Z_{EFFn,SOLL}(BEWEG_{SOLL,RM,AKTk}) between the robot manipulator RM and the body part KT during execution of the target movement, as well as associated permissible deviations: ΔL_{KT,SOLL,AKTk,erlaubt}, ΔBEWEG_{SOLL,AKTk,erlaubt}, ΔINT_{SOLL,AKTk,erlaubt}(BEWEG_{SOLL,RM,AKTk}), and ΔZ_{EFFn,SOLL,erlaubt}(BEWEG_{SOLL,RM,AKTk}); and
controlling the robot manipulator RM upon specification of an activity AKTₖ, as a function of: Z_{RM}(t), L_{KT,AKTk}(t), KW(t), L_{KT,SOLL,AKTk}(t), BEWEG_{SOLL,AKTk}, ΔL_{KT,SOLL,AKTk,erlaubt}, ΔBEWEG_{SOLL,AKTk,erlaubt}, ΔINT_{SOLL,AKTk,erlaubt}(BEWEG_{SOLL,AKTk}), and ΔZ_{EFFn,SOLL,erlaubt}(BEWEG_{SOLL,AKTn}), wherein, as soon as a current movement of the robot manipulator RM determined from the current states Z_{RM}(t) or a currently detected wrench KW(t) or a variable derived therefrom or a current state Z_{EFF}(t) of the effector EFFₙ currently arranged on the robot manipulator RM exceeds a correspondingly predetermined and/or learned permissible deviation, the robot manipulator RM and the effector EFFₙ currently arranged thereon are controllably placed in a safe state SZ_{RM} (AKTₙ), SZ_{EFFn}(AKTₙ) as a function of the currently selected activity AKTₙ, and furthermore the robot manipulator RM and the effector EFFₙ currently arranged thereon are controlled in such a way that images and/or medical parameters and/or samples ascertained while the activity AKTₖ is taking place are provided for further evaluation at a respective interface.

13. The method according to claim 12, wherein the method comprises:
including individual medical information INFO_{PERS} about the person to be examined via a first data interface, wherein the medical information INFO_{PERS} comprises one or more items from a following list:
pre-existing illnesses;
allergies;
familial illnesses;
current medications;
medical risk factors;
previous diagnoses; current diagnostic query; and
specifying via an analysis unit one or more current activities AKTₖ to be carried out in succession, depending on the individual medical information INFO_{PERS} provided: AKTₖ-AKTₖ(INFO_{PERS}).

14. The method according to claim 13, wherein the first data interface comprises a card reader configured to read a chip card, wherein the method comprises storing the individual medical information INFO_{PERS} on the chip card using the card reader.

15. The method according to claim 12, wherein the method comprises transmitting via a second data interface recorded medical parameters and/or image data to a computer in a data network.

16. The method according to claim 15, wherein the method comprises receiving via the second data interface data from the computer or a control unit, wherein the data are output at an optical output unit and/or acoustic output unit and/or which enable the robot manipulator RM to be controlled from the computer.

17. The method according to claim 12, wherein the robot manipulator RM is integrated into a sealable, climate-controlled room which includes a mechanism to kill microbiological germs in the room and on objects located therein, wherein the method comprises automatically activating the mechanism after detection of the medical status of the person has ended, once the person has left the room, no other person is in the room, and the room is closed.

18. The method according to claim 17, wherein the mechanism comprises a UV light source and/or chemicals capable of being spread into the room and/or an ultrasound source.

19. The method according to claim 12, wherein the robot manipulator RM is arranged in a room separated from the person by a partition, wherein the partition includes an opening, wherein the method comprises controlling the robot manipulator RM such that it only guides a distal end of a respective swab or a respective applicator stick or a respective swab spatula or a respective biopsy needle through the opening into the person's body part KT to be examined, in order to get a sample of the microbiological material of the person.

20. The method according to claim 19, wherein the opening on a side of the partition facing away from the room includes a contact device for supported and/or guided contact of the body part to be examined.

21. The method according to claim 12, wherein the selected areas of the person include at least one of external auditory canal, pharynx, specified skin areas, joints, eyes, feet, hands, head, neck, abdomen, and back.
